(19) United States
(12) Patent Application Publication
(10) Pub. No.: US 2005/0064501 A1
(43) Pub. Date: Mar. 24, 2005

(54) Medicaments comprising inhibitors of the cell volume-regulated human kinase h-sgk

(75) Inventors: Florian Lang, Tubingen (DE); Siegfried Waldegger, Hamburg (DE); Carsten Wagner, Tubingen (DE); Stefan Broer, Reutlingen-Ohmenhausen (DE); Karin Klingel, Rottenburg (DE)

Correspondence Address: FOLEY AND LARDNER;SUITE 500, 3000 K STREET NW, WASHINGTON, DC, 20007, US

(73) Assignee: PROF. DR. MED. F. LANG

(21) Appl. No.: 10/984,945

(22) Filed: Nov. 10, 2004

Related U.S. Application Data
(62) Division of application No. 09/959,235, filed on Feb. 19, 2002.

(30) Foreign Application Priority Data
Apr. 20, 1999 (DE) 19917 990.5

(51) Int. Cl. C12Q 1/68

(52) U.S. Cl. 435/6

(57) ABSTRACT

The present invention relates to medicaments comprising inhibitors or activators of the cell volume-regulated human kinase h-sgk. Such medicaments are suitable for the therapy of pathological states in which an increased or reduced expression of h-sgk is found.

### CROSS REFERENCE TO RELATED APPLICATIONS

This application is a divisional of U.S. application Ser. No. 09/959,235, filed Feb. 19, 2002, which is a national stage of International application number PCT/EP00/03578, filed Apr. 19, 2000, which claims priority to DE 199 17 990.5, filed Apr. 20, 1999, all of which are incorporated in their entirety by reference.

### BACKGROUND OF THE INVENTION

The present invention relates to medicaments comprising inhibitors or activators of the cell volume-regulated human kinase h-sgk. Such pharmaceuticals are suitable for the therapy of pathological states in which an increased or reduced expression of h-sgk is found. EP-0 861 896 has already, described h-sgk and processes for its preparation, and the contents thereof are expressly intended also to form part of the present description.

Definitions of Terms:

h-sgk: human serum and glucocorticoid dependent kinase (serine/threonine kinase)
ENaC: epithelial Na⁺ channel
MDEG: mammalian degenerin (Waldmann, R., Lazdunski, M. (1998) Current Opinion in Neurobiology 8: 418-424); a synonymous term is “BNC” (brain Na⁺ channel)
TGFβ₁: tumor growth factor β₁
NKCC: Na⁺, K⁺, 2Cl⁻ cotransporter
HEPES: [4-(2-hydroxyethyl)piperazino]ethanesulfonic acid
SEM: standard error of mean
Trans-dominant inhibitory kinase: h-sgk modified by mutation: lysine in position 127 has been replaced by arginine (K127R); the mutation is located in the catalytic region and suppresses the catalytic function of the kinase.

### SUMMARY OF THE INVENTION

An increased expression of h-sgk is often found in diabetes mellitus, arteriosclerosis, Alzheimer's disease, cirrhosis of the liver, Crohn's disease, fibrosing pancreatitis, pulmonary fibrosis and chronic bronchitis. The increased production of h-sgk can be explained by stimulation of expression by TGFβ₁ (FIG. 1). Fibrotic disorders are caused by increased formation and reduced breakdown of matrix proteins. Both are effects of TGFβ₁. Increased expression of the matrix proteins in fibroblasts can be suppressed by inhibiting the NKCC with furosemide (FIG. 2). It has to date been unclear whether the increased expression of h-sgk is only a consequence or is the cause of the disorder.

Surprising findings now prove h-sgk activates Na⁺, K⁺, 2Cl⁻ cotransport (FIG. 3). It can be concluded from this that stimulation of NKCC by h-sgk induces fibrosis. Besides Na⁺, K⁺, 2Cl⁻ cotransport, h-sgk also activates ENaC (FIGS. 4 and 5) and MDEG.

The stimulating effect of h-sgk on ENaC can be suppressed by kinase inhibitors such as, for example, staurosporine (Sigma, D-82041 Deisenhofen) or chelerythrine (Sigma, loc. cit.) (FIG. 4). In addition, the effect of h-sgk on ENaC can be suppressed, for example, by. trans-dominant inhibitory kinase (FIG. 5). Inhibitors of h-sgk such as staurosporine, chelerythrine or other kinase inhibitors might therefore be employed in the therapy of the abovementioned disorders. Generally suitable for this purpose are all known kinase inhibitors. Kinase inhibitors are also commercially available in many cases, for example from Calbiochem-Novabiochem GmbH, Listweg 1, D-65812 Bad Soden (see “1998 General Catalog”). Further kinase inhibitors can be obtained from other commercial and noncommercial sources known to the skilled worker.

### BRIEF DESCRIPTION OF DRAWINGS

Figure Legends:

FIG. 1: Stimulation of h-sgk expression by TGFβ₁:

The expression of h-sgk is stimulated by TGFβ₁. The effect of TGFβ₁ after 0.5 to 6 h is shown (top). The phorbol ester PDD (4-alpha-phorbol 12,13-didecanoate; stimulates protein kinase C) and the Ca⁺⁺ ionophore ionomycin (Sigma, loc. cit; increases the intracellular Ca⁺⁺ concentration) likewise stimulate h-sgk expression (below).

FIG. 2: Stimulation of biglycan expression by TGFβ₁:

The expression of biglycan (B) is stimulated by osmotic swelling of cells (hypo=h, top left) and by TGFβ₁ (top right). The effect of TGFβ₁ on biglycan expression is almost completely suppressed in the presence of the NKCC inhibitor bumetanide (b) (control=c).

FIG. 3: Stimulation of the NKCC by h-sgk:

The uptake which can be inhibited by furosemide of ²²Na⁺ in oocytes [uptake (nmol/20 min/oocyte)=u] which express the NKCC is massively stimulated by h-sgk. NKCC-injected oocytes do not show a higher Na⁺ influx than uninjected oocytes (n.i.). This Na⁺ influx is not inhibited by the NKCC inhibitor furosemide (═F) (top). Expression of h-sgk alone does not lead to stimulation of the Na⁺ influx. Coexpression of h-sgk with NKCC leads to a large increase in the Na⁺ influx, and this increase is completely suppressed by furosemide (below).

FIG. 4: Stimulation of the ENaC by h-sgk:

The current through the ENaC (I) increases massively through coexpression with h-sgk. Treatment of the oocytes with the kinase inhibitors staurosporine (S) or chelerythrine (C) suppresses the activation of the Na⁺ channel by h-sgk.

FIG. 5: The stimulation of the ENaC by h-sgk can be reversed by coexpression of the trans-dominant inhibitory kinase:

oocytes expressing ENaC and h-sgk simultaneously show very much larger currents (I) than do oocytes expressing only the ENaC. Coexpression of the trans-dominant inhibitory kinase suppresses the stimulation of the ENaC by h-sgk.

FIG. 6: Inhibition of the MDEG by h-sgk:

The current through the MDEG (I) increases with the duration of the incubation [day (T) 1-4]. The current is completely suppressed by coexpression with h-sgk (peak=p; plateau=pl).

### DETAILED DESCRIPTION OF INVENTION

Expression of h-sgk is increased in an epileptic seizure. The functional data we have found show that the effects are suitable for reducing the excitability of neurons because activation of NKCC leads to a reduction in the extracellular K⁺ concentration, which is followed by hyperpolarization and thus inhibition of the activity of neurons. In addition, the inhibition of MDEG ought to inhibit neuronal excitability. Accordingly, kinase activators which cross the blood-brain barrier might be employed successfully for epileptic seizures. Conversely, kinase inhibition with drugs crossing the blood-brain barrier might increase attentiveness and learning ability. Kinase activators have moreover been known to the skilled worker for a lengthy period, among which the protein kinase C activators are particularly of interest (see, for example, Calbiochem-Novabiochem 1998 General Catalog, loc. cit.). Further kinase activators can be obtained from other commercial and noncommercial sources known to the skilled worker.

Since the Na⁺, K⁺, 2Cl⁻ cotransport and the Na⁺ channel are crucial for renal Na⁺ absorption and an increased renal Na⁺ absorption is associated with hypertension, it must be assumed that increased expression of the kinase leads to hypertension and reduced expression of the kinase leads to hypotension.

The present invention thus also relates to the use of inhibitors of h-sgk for producing medicaments for the treatment of diabetes mellitus, arteriosclerosis, Alzheimer's disease, cirrhosis of the liver, Crohn's disease, fibrosing pancreatitis, pulmonary fibrosis, chronic bronchitis, radiation fibrosis, scleroderma, cystic fibrosis and other fibrosing disorders, and for the therapy of essential hypertension. Medicaments comprising inhibitors or activators of h-sgk can additionally be employed to regulate neuronal excitability. It is particularly advantageous to use the inhibitors staurosporine or chelerythrine and their analogs.

Results

Diabetic Kidney:

Expression of h-sgk in the normal kidney is only low. A few cells in the glomerulus, late proximal and distal tubule show distinct h-sgk expression. In contrast to this, cells with massive h-sgk expression accumulate in the diabetic kidney.

Arteriosclerosis:

Cells massively expressing h-sgk are frequently found in the walls of arteriosclerotic vessels.

Alzheimer's Disease:

Only a few cells expressing h-sgk are found in the normal brain. These cells are probably oligodendroglial cells. The number of h-sgk-expressing cells is significantly increased in brains with Alzheimer's disease.

Cirrhosis of the Liver:

Only Kupffer cells express h-sgk in the normal liver. However, in cirrhosis of the liver the tissue is dotted with h-sgk-expressing cells.

Crohn's Disease:

In normal intestinal tissue, h-sgk is expressed exclusively in the enterocytes. However, in Crohn's disease, the kinase is also found in connective tissue.

Fibrosing Pancreatitis:

In the normal pancreas, h-sgk is found in acinar cells and in duct cells. A few h-sgk-expressing mononuclear cells are found around the pancreatic ducts. There is a marked increase in expression of the kinase in fibrosing pancreatitis.

Pulmonary Fibrosis and Chronic Bronchitis:

Massive expression of h-sgk is observed in pulmonary fibrosis and chronic bronchitis.

Stimulation of h-sgk Expression by TGFβ₁:

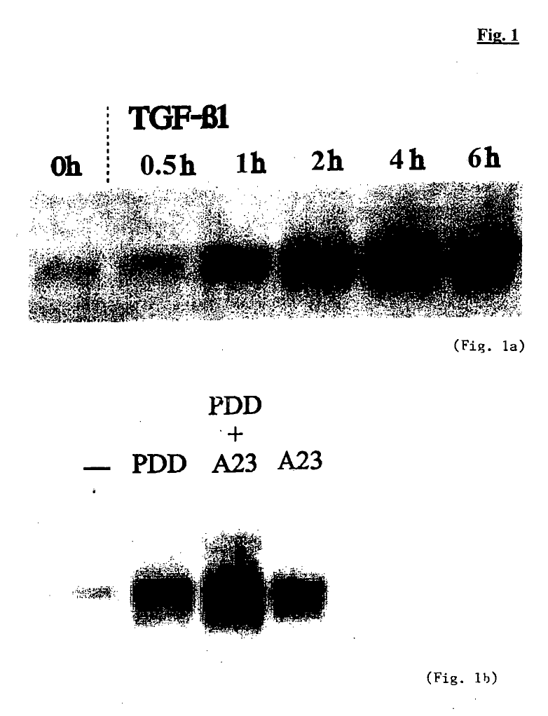

The expression of h-sgk is stimulated by TGFβ₁ (FIG. 1). Since TGFβ₁ is produced in fibrotic/inflamed tissue, this finding explains the increased expression of h-sgk in inflamed tissue.

TGFβ₁ stimulates the expression of the matrix protein biglycan, an effect which is suppressed by the NKCC inhibitor furosemide:

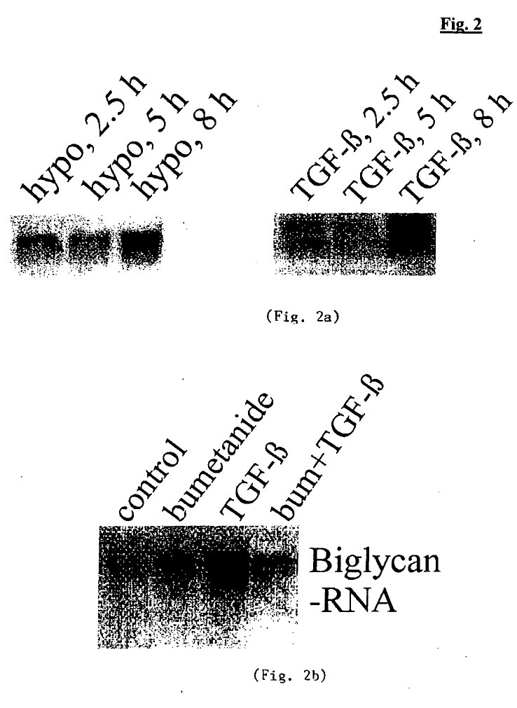

TGFβ₁ stimulates the expression of biglycan. In the presence of the NKCC inhibitor furosemide, the effect of TGFβ₁ on biglycan expression is completely suppressed. Thus activation of NKCC is a precondition for the fibrotic effect of TGFβ₁. (FIG. 2).

Stimulation of NKCC by h-sgk:

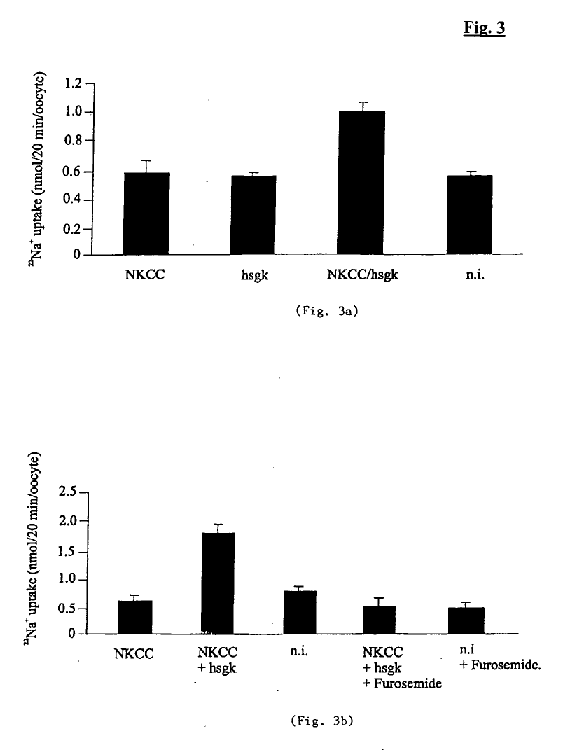

The significance of the increased expression of the kinase in fibrotic tissue might be manifold and not causally connected with the fibrosis. However, experiments with the two-electrode voltage clamp have shown that the activity of NKCC is massively stimulated by h-sgk (FIG. 3). In view of the furosemide sensitivity of biglycan synthesis, this finding unambiguously demonstrates a causal role of h-sgk in fibrosis.

Stimulation of ENaC by h-sgk:

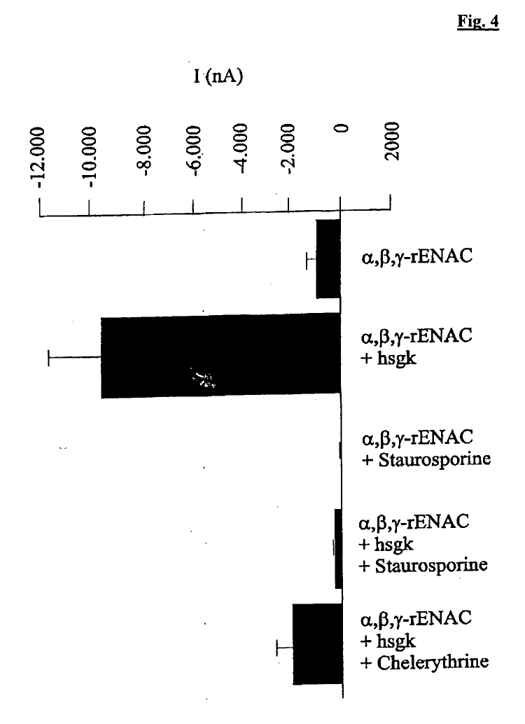

This effect can be suppressed by the kinase inhibitors staurosporine and chelerythrine. As FIG. 4 shows, there is a massive increase in the current with ENaC through coexpression with h-sgk. The kinase therefore stimulates ENaC. The kinase inhibitors staurosporine and chelerythrine are able completely to suppress the activation of ENaC by h-sgk.

Stimulation of epithelial ENaC by h-sgk can be reversed by coexpression of the trans-dominant inhibitory kinase h-sgk:

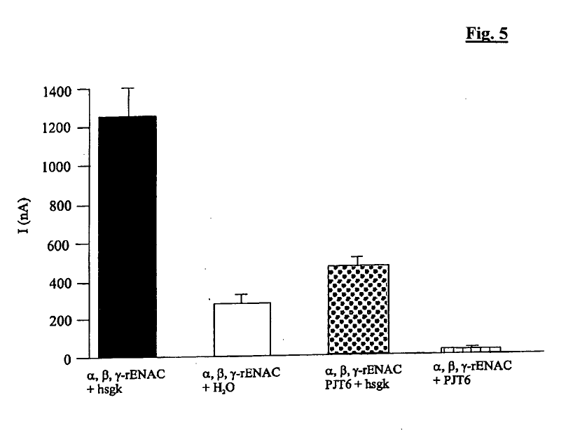

As FIG. 5 shows, the stimulating effect of h-sgk coexpression on the ENaC-mediated Na⁺ current can be suppressed by coexpression of a trans-dominant inhibitory kinase. This trans-dominant inhibitory kinase (compare with “definitions of terms”) is modified on the catalytic unit in such a way that it can no longer display its function. However, since it binds to the substrate it displaces the active kinase and thus suppresses its effects. The trans-dominant inhibitory kinase not only suppresses the increase in ENaC activity due to exogenous h-sgk but evidently also suppresses the stimulation by endogenous h-sgk.

MDEG is completely blocked by coexpression with h-sgk:

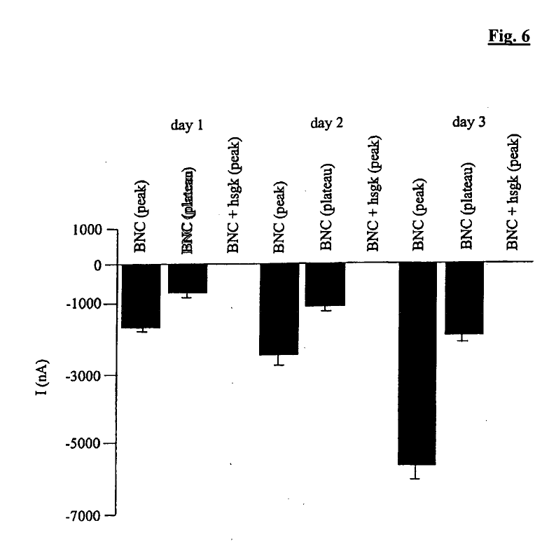

As FIG. 6 shows, expression of MDEG in oocytes induces a strong Na⁺ current which is activated by lowering the extracellular pH. The channel is completely blocked by coexpression with h-sgk. It must be concluded from this that h-sgk inhibits neuronal excitability.

### EXAMPLES

### Example 1

### In Situ Hybridization

Tissue from normal pancreas, liver, vessels, brain, lung, kidney and intestine, and tissue with diabetic nephropathy, arteriosclerosis, Alzheimer's disease, cirrhosis of the liver, Crohn's disease, fibrosing pancreatitis and pulmonary fibrosis was embedded in paraffin in 4% paraformaldehyde/0.1 M sodium phosphate buffer (pH 7.2) for 4 hours. Tissue sections were dewaxed and hybridized as described previously (Kandolf, R., D. Ameis, P. Kirschner, A. Canu, P. H. Hofschneider, Proc. Natl. Acad. Sci. USA 84: 6272-6276, 1987; Hohenadl, C., K. Klingel, J. Mertsching, P. H. Hofschneider, R. Kandolf., Mol. Cell. Probes 5: 11-20, 1991; Klingel, K., C. Hohenadl, A. Canu, M. Albrecht, M. Seemann, G. Mall, R. Kandolf, Proc. Natl. Acad. Sci. USA, 89: 314-318, 1992).

The hybridization mixture contained either ³⁵S-labeled sense RNA coding for h-sgk or ³⁵S-labeled antisense RNA complementary to the latter RNA (500 ng/ml of each) in 10 mM Tris-HCl, pH 7.4; 50% (vol/vol) deionized formamide; 600 mM NaCl; 1 mM EDTA; 0.2% polyvinylpyrrolidone; 0.02% Ficoll; 0.05% calf serum albumin; 10% dextran sulfate; 10 mM dithiothreitol; 200 μg/ml denatured sonicated salmon sperm DNA and 100 μg/ml rabbit liver tRNA.

Hybridization with RNA probes was carried out at 42° C. for 18 hours. The slides were washed as described (Hohenadl et al., 1991; Klingel et al., 1992), and then incubated in 2× standard sodium citrate at 55° C. for 1 hour. Unhybridized single-stranded RNA probes were digested by RNase A (20 μg/ml) in 10 mM Tris-HCl, pH 8.0/0.5 M NaCl at 37° C. for 30 min. Tissue samples were then autoradiographed for three weeks (Klingel et al., 1992) and stained with hematoxylin/eosin.

### Example 2

### Transcriptional Regulation of biglycan and h-sgk

Cells were cultivated in RPMI/5% CO₂/10 mM glucose at 37° C., pH 7.4, supplemented with 10% (vol/vol) fetal calf serum (FCS). The cells were grown to 90% confluence and then homogenized in TRIZOL (GIBCO/BRL) (about 0.4×10⁶ per sample). Total RNA was prepared in accordance with the manufacturer's instructions. Northern blots were fractionated by electrophoresis through 10 g/l agarose gels with 15 or 20 μg of total RNA with separate control in the presence of 2.4 mol/l formaldehyde. RNA was transferred by vacuum (Appligene Oncor Trans DNA Express Vacuum Blotter, Appligine, Heidelberg, Germany) to positively charged nylon membranes (Boehringer Mannheim, Germany) and crosslinked under ultraviolet light (UV Stratalinker 2400, Stratagene, Heidelberg, Germany). Hybridization was carried out over night with DIG-Easy-Hyb (Boehringer Mannheim) at a probe concentration of 25 μg/l at 50° C. The digoxigenin (DIG)-labeled probes were produced by PCR as described in detail earlier (Waldegger et al. (1997) PNAS 94: 4440-4445). For the autoradiography, the filters were exposed to an X-ray film (Kodak) for an average of 5 min.

### Example 3

### Two-Electrode Voltage Clamp and Tracer Flux Experiments

Dissection of Xenopus laevis, and the obtaining and treatment of the oocytes has been described in detail earlier (Busch et al. 1992). The oocytes were each injected with 1 ng of cRNA of NKCC, ENaC or MDEG with or without simultaneous injection of h-sgk. It was possible to carry out two-electrode voltage and current clamp experiments 2-8 days after the injection. Na⁺ influx which could be inhibited by furosemide through the NKCC was measured by the ²²Na⁺ uptake, which was determined with a scintillation counter, into the oocytes. Na⁺ currents (ENaC) were filtered at 10 Hz and recorded with a pen recorder. The experiments were normally carried out on the second day after cRNA injection. The bath solution contained: 96 mM NaCl, 2 mM KCl, 1.8 mM CaCl₂, 1 mM MgCl₂ and 5 mM HEPES at pH 7.5 and the holding potential was −50 mV. The pH was adjusted by titration with HCl or NaOH in all the experiments. The flow rate of the bath liquid was set at 20 ml/min, which ensured a complete change of solution in the measurement chamber within 10-15 s. All the data are stated in the form of arithmetic means ±SEM.

1. A method of diagnosing hypertension or at least one of the disorders from the group of fibrosing pancreatitis, radiation fibrosis, scleroderma, cystic fibrosis, chronic bronchitis and epilepsy comprising detecting increased expression of the human cell volume-regulated kinase h-sgk.

2. The method according to claim 1, wherein the detecting is by in situ hybridization or Northern blot.

3. The method according to claim 2, wherein the in situ hybridization or Northern blot is performed with a hybridization mixture which comprises an antisense RNA complementary to the RNA coding for h-sgk.

4. The method according to claim 1, wherein the method is for diagnosing hypertension.

5. A method of diagnosing hypotension comprising detecting decreased expression of the human cell volume-regulated kinase h-sgk.

6. The method according to claim 5, wherein the detecting is by in situ hybridization or Northern blot.

7. The method according to claim 6, wherein the in situ hybridization or Northern blot is performed using a hybridization mixture which comprises an antisense RNA complementary to the RNA coding for h-sgk.

8. A kit comprising a means for detecting increased or decreased expression of the human cell volume-regulated kinase h-sgk.

9. The kit according to claim 8, wherein the means is an antisense RNA complementary to the RNA coding for h-sgk.

10. The kit according to claim 9, wherein the antisense RNA is labeled.
